(19) United States
(12) Patent Application Publication
(10) Pub. No.: US 2019/0292687 A1
(43) Pub. Date: Sep. 26, 2019

(54) NATURAL POLYMERIC NANOFIBER AND MANUFACTURING METHOD THEREOF

(71) Applicant: WIZDOM INC., Las Vegas, NV (US)

(72) Inventors: Chih Hsiang Fang, Taipei City (TW); Yung Lung Liu, Kaohsiung City (TW)

(21) Appl. No.: 15/928,923

(22) Filed: Mar. 22, 2018

(51) Int. Cl. D01F 4/00; D01D 5/00; D01F 11/00; D01F 1/10; C12P 21/00

(52) U.S. Cl. CPC D01F 4/00; D01D 5/0046; D10B 2509/00; D01F 1/10; C12P 21/00; D01F 11/00

(57) ABSTRACT

The present invention provides a natural polymeric nanofiber, which is made by a natural polymeric solution under an applied field control spinning process, wherein the natural polymeric solution comprises: a natural polymeric material, an inorganic salt and/or an enzyme; the present invention further provides a method for preparing the natural polymeric nanofibers. The nature polymeric nanofibers of the present invention have a smaller fiber diameter, and have higher safety and practicability.

### FIELD OF THE INVENTION

The present invention is related to a type of nanofiber and manufacturing method thereof, more particularly to a type of natural polymeric nanofiber and manufacturing method thereof.

### DESCRIPTION OF THE RELATED ART

Nanofibers are nanometer-sized fibers with fiber fineness ranging from tens to hundreds of nanometers, and their size can be adjusted by different manufacturing methods. Due to the high surface area of nanofibers, the nets made of nanofibers have a low pore size distribution and high porosity. The application of nanofibers in biomedical field has been growing gradually. Currently, the common nanofibers are made by electrospinning.

Electrospinning is a method of using high voltage electrostatic polymer solution to prepare microfibers or nanofibers. The materials used by traditional electrospinning are artificial polymers and organic solvents. In recent years, natural polymer-based nanofibers with special structure are available thanks to the modification to the natural polymers and improvement of electrospinning equipment. However, chemical modification of natural polymers is still required for the aforementioned natural polymeric nanofibers. Such modification changes the structure of the natural polymers (such as proteins, . . . etc.), and thus the application to industry, medical treatment, food and traditional processing is limited, especially in food and medical industry where organic solvents, modified and degenerated polymers or proteins are prohibited. Therefore, it is necessary to develop natural polymeric nanofibers applicable to nanospinning without the involvement of organic solvents or physicochemical modification.

### SUMMARY OF THE INVENTION

In order to solve the above problem, the present invention provides a natural polymeric nanofiber, which is made from a natural polymer solution under a spinning process controlled by an applied field force, wherein the natural polymeric solution comprises: a natural polymeric material, an inorganic salt, and/or an enzyme.

In a preferable embodiment, the natural polymeric material is selected from but not limited to the group consisting of: gelatin, collagen, hyaluronic acid, alginate and chitosan.

In a preferable embodiment, the inorganic salt is selected from but not limited to the group consisting of: hydroxyapatite (Hap), tricalcium phosphate (TCP), dicalcium phosphate (DCP), dicalcium phosphate dehydrate (DCPD), tetracalcium phosphate (TTCP), carbonates, nitrates, sulfates, calcium, potassium, sodium and magnesium.

In a preferable embodiment, the enzyme is but not limited to the group consisting of: transglutaminase, lipase, peptidase, sortase, oxidoreductase, tyrosinase, polyphenoloxidase (PPO), laccase, peroxidase, lysyl oxidase, or amine oxidase.

In a preferable embodiment, the applied field force is but not limited to the group consisting of: electric field, magnetic field or gravity.

In a preferable embodiment, the average fineness of the natural polymeric nanofiber is between 10 nm to 900 nm.

In a preferable embodiment, the concentration of the natural polymeric material in the natural polymeric solution is between 0.5% (W/V) to 100% (W/V).

In a preferable embodiment, the concentration of the inorganic salt is between 0.5% (W/V) to 100% (W/V).

Another aspect is to provide method for preparing natural polymeric nanofibers, comprising: (a) providing a natural polymer solution, comprising a natural polymeric material, an inorganic salt and/or an enzyme; and (b) applying a spinning process controlled by an applied field force to the natural polymeric solution to obtain the natural polymeric nanofibers.

In a preferable embodiment, the natural polymeric material used in the method is selected from the group consisting of: gelatin, collagen, hyaluronic acid, alginate and chitosan.

In a preferable embodiment, the inorganic salt used in the method is selected from the group consisting of: hydroxyapatite (Hap), tricalcium phosphate (TCP), dicalcium phosphate (DCP), dicalcium phosphate dehydrate (DCPD), tetracalcium phosphate (TTCP), carbonates, nitrates, sulfates, calcium, potassium, sodium and magnesium.

In a preferable embodiment, the enzyme used in the method is transglutaminase.

In a preferable embodiment, the applied field force used in the method is electric field, magnetic field or gravity.

In a preferable embodiment, the method further comprises: (c) exposing the natural polymeric nanofibers to an enzyme for external crosslinking.

Another aspect of the present invention is to provide a use of natural polymeric nanofiber of the present invention, wherein the natural polymeric nanofiber is used as a biomedical material.

Compared with the prior art, the present invention can form nanofibers with uniform fiber fineness and morphology without changing the physicochemical properties of natural polymers and the use of organic solvents. Because artificial polymers and organic solvents are not used, the safety is significantly improved, and the scope of application is expanded, especially in the food and biomedical industries.

### BRIEF DESCRIPTION OF THE DRAWINGS

FIG. 1. Photo of the natural polymeric nanofibers made in accordance with the description of Embodiment 1.

FIG. 2. Photo of the natural polymeric nanofibers made in accordance with the description of Embodiment 2.

FIG. 3. Photo of the natural polymeric nanofibers made in accordance with the description of Embodiment 3.

FIG. 4. Photo of the natural polymeric nanofibers made in accordance with the description of Embodiment 4.

FIG. 5. Photo of the natural polymeric nanofibers made in accordance with the description of Embodiment 5.

FIG. 6. Photo of the natural polymeric nanofibers made in accordance with the description of Embodiment 6.

### DETAILED DESCRIPTION OF THE PREFERRED EMBODIMENTS

Unless defined otherwise, all technical and scientific terms used herein have the same meaning as commonly understood by one of ordinary skill in the art to which this invention belongs. As used in this application, the following terms have the following meanings.

“A”, “ₐn”, “one” and “the” include plural referents unless the content in the context otherwise requires. The terms used herein, such as “a”, “the”, “one or more”, “plural” and “at least one,” are interchangeable.

The terms “comprising”, “including”, “containing” and “having” as used herein are also interchangeable without limitation.

In addition, “and/or” as used herein is used to refer specifically to one or all of two particular features or compositions. Thus, the term “and/or” is used to convey that a sentence such as “A and/or B” means “A and B”, “A or B”, “(alone) A” or “(alone) B”. Similarly, the use of the term “and/or” in expressions such as “A, B and/or C” includes the meanings described below: A, B and C; A, B or C; A or C; A or B; B or C; A and C; A and B; B and C; (alone) A; (alone) B; (alone) C.

The natural polymeric nanofiber of this invention is prepared from a natural polymer solution under a spinning process controlled by an applied field force, wherein the natural polymer solution includes a natural polymeric material, an inorganic salt and/or an enzyme.

The term “natural polymeric material” used herein refers to a polymer existing in nature rather than artificially synthesized, including but not limited to gelatin, collagen, hyaluronic acid, alginic acid, chitosan and the derivatives of the aforementioned natural polymers. In a preferred embodiment, the average molecular weight of the natural polymeric material is between 10,000 to 200,000, more preferably between 15,000 to 150,000, and even more preferably between 20,000 to 100,000.

The natural polymer solution referred to herein is a solution comprising the said natural polymeric material dissolved in a solvent, wherein the solvent can be any polar solvent. In a preferred embodiment, the polar solvent is water. The concentration of the natural polymeric material in the natural polymer solution is between 0.1% (W/V) to 50% (W/V), preferably about 0.5% (W/V) to 40%, even more preferably 1% (W/V) to 30% (W/V), such as 10% (W/V), 15% (W/V), and 20% (W/V).

The natural polymer solution referred to herein further comprises an inorganic salt. The inorganic salt referred to herein is an inorganic salt that can modify the chargeability of the natural polymeric material, including but not limited to hydroxyapatite, tricalcium phosphate, dicalcium phosphate, dicalcium phosphate dihydrate, phosphoric acid tetracalcium, carbonate, nitrate, sulfate, calcium, potassium, sodium and magnesium. The concentration of the inorganic salt in the natural polymer solution is between 0.1% (W/V) to 50% (W/V), preferably between 0.5% (W/V) to 40%, even more preferably between 1% (W/V) to 30% (W/V), such as 10% (W/V), 15% (W/V) and 20% (W/V).

The natural polymeric material and the inorganic salt in the above natural polymer solution are mixed for 1 to 96 hours, preferably 12 to 48 hours, more preferably 24 to 36 hours, such as 24, 26, 28, 30, 32, 34 and 36 hours. The natural polymer solution referred to herein may or may not comprise an enzyme. In a preferred embodiment, the natural polymer solution comprises an enzyme, wherein the enzyme enables internal crosslink of the natural polymer, which in turn enhances the strength of the natural polymeric nanofiber. The enzyme that can be used is preferably, but not limited to, transglutaminase, lipase, peptidase, sortase, oxidoreductase, tyrosinase, polyphenoloxidase (PPO), laccase, peroxidase, lysyl oxidase, or amine oxidase, and the concentration can be used is between 1% (W/V) to 100% (W/V).

The method for preparing the natural polymeric nanofiber of the present invention comprises (a) providing a natural polymer solution comprising: a natural polymeric material, an inorganic salt and/or an enzyme; and (b) applying a spinning process controlled by an applied field force to the natural polymeric solution to obtain the natural polymeric nanofiber.

The “spinning process controlled by an applied field force” referred in the preparation method of the present invention means that the natural polymer solution is loaded into a spinning device for a spinning process, and an applied field force, including but not limited to electric field, magnetic field, gravity, is applied to the process to drive the anions and cations in the natural polymer solution, which in turn drives the natural polymeric material to form a nanofiber. According to a preferred embodiment of the present invention, the spinning process controlled by an applied field force is a discharge spinning process with an applied voltage, wherein the applied voltage ranges from 1 μV to 1000 kV, preferably between 1 kV to 70 kV, more preferably between 30 kV to 50 kV. In addition, the distance between the spinneret and the collection area of the discharge spinning process is between 1 cm to 30 cm, and the discharge rate of the discharge spinning process may be 10 μl/min or higher.

The manufacturing method of the present invention may further comprise an external crosslinking step, which is the crosslink resulting from the contact between the natural polymeric nanofiber obtained above and an ezymeric crosslinking agent to improve the strength of the natural polymeric nanofiber and prolong the degradation time of the natural polymeric nanofiber. The crosslinking time can range from 1 to 96 hours, preferably 24 to 48 hours, such as 24, 26, 28, 30, 32, 34, 36, 3, 40, 42, 44, 46 and 48 hours. The term “contact” mentioned above referred to any means by which the ezymic crosslinking agent interacts with the natural polymeric nanofiber, including but not limited to soaking the natural polymeric nanofiber in an enzymic crosslinking agent or spreading an enzymic crosslinking agent on the natural polymeric nanofiber.

The average fineness of the natural polymeric nanofibers prepared by the above method is between 10 nm to 500 nm, preferably between 10 nm to 50 nm, for example, 10 nm, 20 nm, 30 nm, 40 nm and 50 nm. Because the natural polymeric nanofiber of the present invention has better fineness and improved safety, it can be more widely applied to food and biomedical fields, such as a nanofiber material for biomedical 3D printing, as compared to artificial polymeric nanofibers.

In order to enable any person skilled in the art to understand and implement the technical content of the present invention, and according to the disclosure of this specification, the scope of the patent application and the drawings, anyone familiar with the art can easily understand the present invention objects and advantages of the invention, the detailed features and advantages of the invention will be described in detail in the examples. The following embodiments are merely illustrative and not intended to limit the scope of the present patent application.

### Embodiment 1

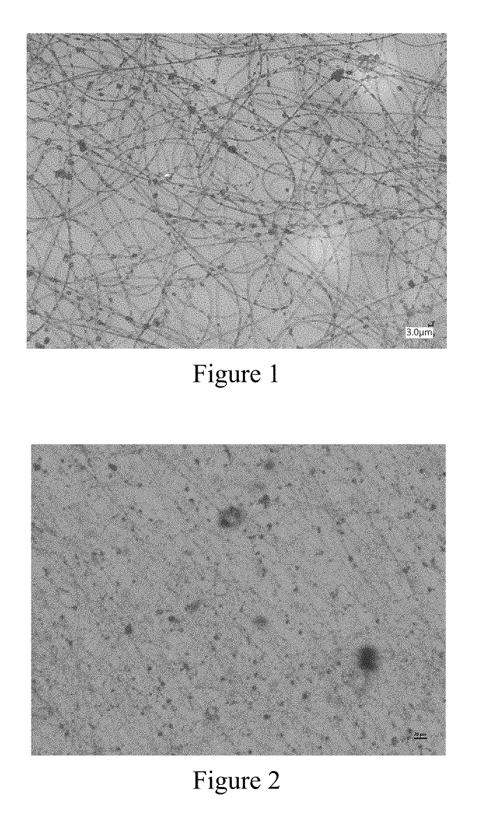

A 20% (w/v) gelatin solution was prepared, and 20% (w/v) hydroxyapatite was added to the solution followed by continuous mix for 24 hours. Later, the solution was loaded to a 10 ml syringe at a controlled temperature of 50° C.±5° C. The height for fiber drawing was 60 mm, the ejection speed was 2 ml/hr, and the voltage of the electrical field was 20 kV. The resulting natural polymer nanofibers are shown in FIG. 1.

### Embodiment 2

The same formulation as in Embodiment 1 was used. The only difference was that a 10 wt % enzymic crosslinking agent was added in Embodiment 2. The resulting natural polymer nanofibers are shown in FIG. 2.

### Embodiment 3

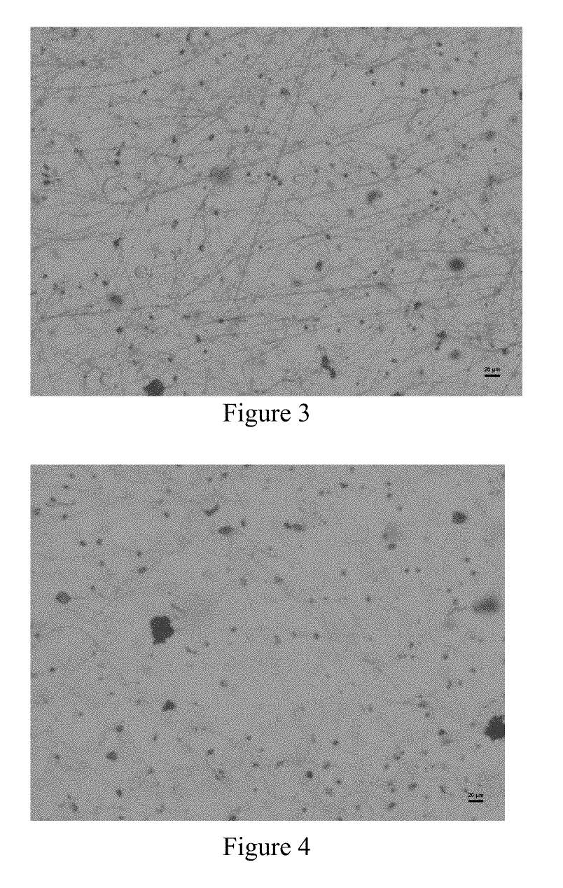

The same formulation as in Embodiment 1 was used except that 20% (w/v) hydroxyapatite was replaced with tricalcium phosphate (TCP) The resulting natural polymer nanofibers are shown in FIG. 3.

### Embodiment 4

The same formulation as in Embodiment 3 was used. The only difference was that a 10 wt % enzymic crosslinking agent was added in Embodiment 4, and the ejection speed and voltage of the electric field were changed to 1.3 ml/hr and 13.5 kV, respectively. The rest was the same. The resulting natural polymer nanofibers are shown in FIG. 4.

### Embodiment 5

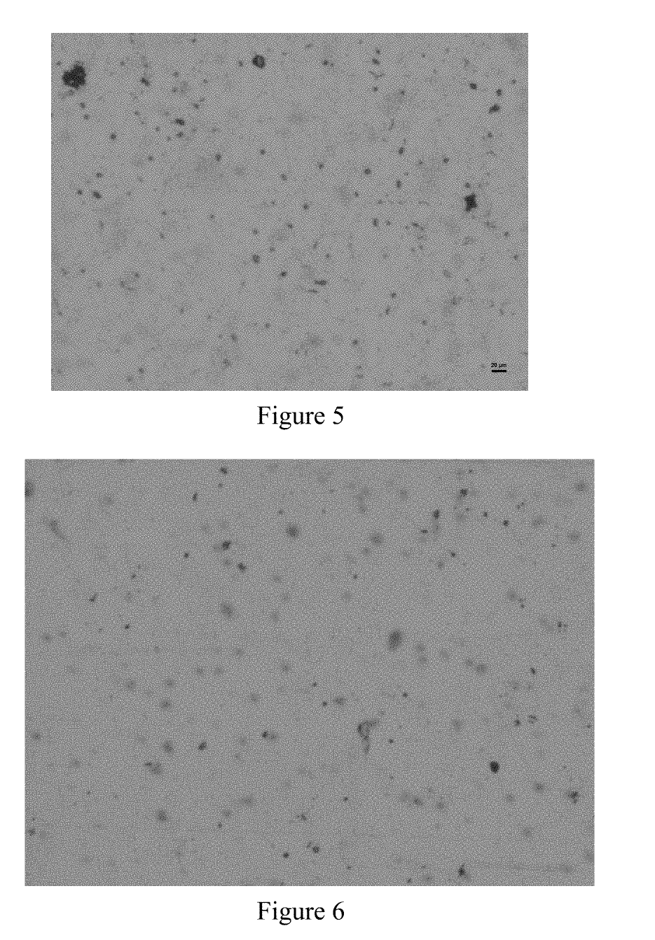

A 5% (w/v) gelatin solution was prepared, and 5% (w/v) TCP was added to the solution followed by continuous mix for 24 hours. Later, the solution was loaded to a 10 ml syringe at a controlled temperature of 50° C.±5° C. The height for fiber drawing was 60 mm, the ejection speed was 0.9 ml/hr, and the voltage of the electrical field was 7 kV. The resulting natural polymer nanofibers are shown in FIG. 5.

### Embodiment 6

The only difference between Embodiment 5 and Embodiment 6 is that 10 wt % enzymic crossing agent was added in Embodiment 6. The rest was the same. The resulting natural polymer nanofibers are shown in FIG. 6.

The above embodiments are merely illustrative of the features of the present invention and are intended to enable those skilled in the art to understand and practice the contents of the present invention rather than to limit the scope of the invention, The equivalent modifications or changes made in the spirit of the invention should still be covered by the following claims.

What is claimed is:

1. A natural polymeric nanofiber, which is made from a natural polymer solution under a spinning process controlled by an applied field force, wherein the natural polymeric solution comprises: a natural polymeric material, an inorganic salt, and/or an enzyme, wherein the concentration of the natural polymeric material in the natural polymeric solution is between 0.5% (W/V) to 50% (W/V), and the concentration of the inorganic salt is between 0.5% (W/V) to 50% (W/V).

2. The natural polymeric nanofiber of claim 1, wherein the natural polymeric material is selected from the group consisting of: gelatin, collagen, hyaluronic acid, alginate and chitosan.

3. The natural polymeric nanofiber of claim 2, wherein the inorganic salt is selected from the group consisting of: hydroxyapatite (Hap), tricalcium phosphate (TCP), dicalcium phosphate (DCP), dicalcium phosphate dehydrate (DCPD), tetracalcium phosphate (TTCP), carbonates, nitrates, sulfates, calcium, potassium, sodium and magnesium.

4. The natural polymeric nanofiber of claim 3, wherein the enzyme is transglutaminase, lipase, peptidase, sortase, oxidoreductase, tyrosinase, polyphenoloxidase (PPO), laccase, peroxidase, lysyl oxidase, or amine oxidase.

5. The natural polymeric nanofiber of claim 4, wherein the applied field force is electric field, magnetic field or gravity.

6. The natural polymeric nanofiber of claim 5, wherein the average fineness of the natural polymeric nanofiber is between 10 nm to 900 nm.

7. The natural polymeric nanofiber of claim 1, wherein the concentration of the natural polymeric material in the natural polymer solution is between 0.1% (W/V) to 100% (W/V).

8. The natural polymeric nanofiber of claim 1, wherein the concentration of the inorganic salt in the natural polymer solution is between 0.1% (W/V) to 100% (W/V).

9. A method for preparing natural polymeric nanofibers, comprising:
(a) providing a natural polymer solution, comprising a natural polymeric material, an inorganic salt and/or an enzyme; and
(b) applying a spinning process controlled by an applied field force to the natural polymeric solution to obtain the natural polymeric nanofibers.

10. The method of claim 9, wherein the natural polymeric material is selected from the group consisting of: gelatin, collagen, hyaluronic acid, alginate and chitosan.

11. The method of claim 11, wherein the inorganic salt is selected from the group consisting of: hydroxyapatite (Hap), tricalcium phosphate (TCP), dicalcium phosphate (DCP), dicalcium phosphate dehydrate (DCPD), tetracalcium phosphate (TTCP), carbonates, nitrates, sulfates, calcium, potassium, sodium and magnesium.

12. The method of claim 9, wherein the method further comprises:
(c) exposing the natural polymeric nanofibers to an enzyme for external crosslinking.

13. The method of claim 12, wherein the enzyme is transglutaminase, lipase, peptidase, sortase, oxidoreductase, tyrosinase, polyphenoloxidase (PPO), laccase, peroxidase, lysyl oxidase, or amine oxidase.

14. The method of claim 13, wherein the applied field force is electric field, magnetic field or gravity.

15. Use of the natural polymeric nanofiber as claimed in claim 1, wherein the natural polymeric nanofiber is used as a biomedical material.
